(19) United States
(12) Patent Application Publication
(10) Pub. No.: US 2005/0172344 A1
(43) Pub. Date: Aug. 4, 2005

(54) Cell cultures from animal models of Alzheimer's disease for screening and testing drug efficacy

(76) Inventors: Ottavio Arancio, New York, NY (US); Paul M. Mathews, Irvington, NY (US); Stephen D. Schmidt, Nutley, NJ (US); Ralph A. Nixon, Tarrytown, NY (US); Fortunato Battaglia, New York, NY (US); Fabrizio Trinchese, Rome (IT); Shumin Liu, Tappan, NY (US)

Correspondence Address: MORGAN & FINNEGAN, L.L.P., 3 World Financial Center, New York, NY, 10281-2101, US

(21) Appl. No.: 10/980,922

(22) Filed: Nov. 3, 2004

Related U.S. Application Data
(63) Continuation-in-part of application No. PCT/US03/13948, filed on May 5, 2003.
(60) Provisional application No. 60/377,735, filed on May 3, 2002.

(51) Int. Cl. A01K 67/027; C12N 5/06

(52) U.S. Cl. 800/12; 435/354

(57) ABSTRACT

The present invention describes a dissociated cell culture system comprising cells of the hippocampus, one of the brain areas affected by Alzheimer's Disease (AD) or amyloid beta-related diseases. This culture system comprises hippocampal neuronal and glial cells from animal models of AD, particularly, but not limited to, double transgenic mice expressing both the human APP mutation (K670N:M671L) (mAPP), and the human PS1 mutation (M146L) (mPS1), and serves as a powerful tool for the screening and testing of compounds and substances, e.g., drugs, for their ability to affect, treat, or prevent AD or β-amyloid-related diseases. The effects of a test substance on the cells in this culture system can be quantitatively assessed to determine if the test substance affects the cells biochemically and/or electrophysiologically, and/or optically, and/or immunocytochemically. The present in vitro culture system is advantageous for AD drug screening, because it is rapid and efficient. By contrast, even in the fastest animal model of AD, pathology does not start before the end of the second month. If such in vivo animal models are used, it is necessary to wait at least the two month time duration or longer to test for drug efficacy for AD treatment or prevention. At the same time the present invention provides a tool for production of amyloid-beta that can be used for electrophysiological, behavioral, and toxicological studies.

This is a continuation-in-part of International Application PCT/US03/13948, filed on May 5, 2003, which claims priority to U.S. application Ser. No. 60/377,735, filed on May 3, 2002.

### FIELD OF THE INVENTION

The present invention relates to improved screening methods which can test, in a fast, efficient and cost effective manner, biological compounds and materials, e.g., drugs, for use in the treatment or prevention of Alzheimer's disease or a beta-amyloid (β-amyloid)-associated disease.

### BACKGROUND OF THE INVENTION

Approximately two million people in the United States suffer from Alzheimer's Disease (AD), which is the most common cause of chronic dementia among the aging population. Neuritic amyloid plaques, neurofibrillary degeneration, and granulovascular neuronal degeneration constitute the histopathologic lesions of Alzheimer's Disease and are found in the brains of elderly people with Alzheimer's dementia. It is estimated that ten percent of individuals older than 65 years of age have mild to severe dementia. The number of such lesions correlates with the degree of intellectual deterioration. This high prevalence, combined with the rate of growth of the elderly segment of the population, make dementia (and particularly AD) one of the most important of the present-day public health concerns.

During the last seven years, excellent opportunities to screen drugs against Alzheimer's Disease have been provided by animal models of the disease. Features of Alzheimer's Disease have already been reproduced in transgenic mouse models of Alzheimer's Disease, for example, transgenic mice in which mutant forms of amyloid precursor protein (APP) and presenilin I (PS1) and II (PS2), two peptides that alter APP processing, are overexpressed (K. Duff et al., Nature, 383:710-713, 1996). Most of these models have been investigated from the behavioral and histopathological point of view. For instance, transgenic mice expressing the wild type APP₇₅₁ produced by Moran et al. (Moran et al., Proc. Natl. Acad. Sci. USA, 92:5341-5, 1995) show deficits in spatial reference and alternation tasks at 12 months of age, with diffuse deposits of β-amyloid, a 40 to 43 amino acid protein derived from the larger (approximately 100 kDa) amyloid precursor protein (APP), and aberrant tau protein expression in the brain. Immunization of the young transgenic APP₇₅₁ animals with β-amyloid 42 peptide prevents the development of amyloid beta plaque formation, neuritic dystrophy, and astrogliosis (Schenk et al., Nature, 400:173-7, 1999).

A different transgenic mouse, which expresses the familial Swedish mutation in APP(K670N:M671L), shows defects in both spatial reference and alternation tasks, together with senile plaques in cortical and limbic structures, and an increase of the levels of β-amyloid, at 9 to 10 months of age. (K. Hsiao et al., Science, 274:99-102, 1996). Recently, it has been shown that these animals have impairment of long-term potentiation (LTP), a type of plasticity that is a synaptic model of learning and memory, at 9-10 months of age (P. F. Chapman et al., Nat Neurosci., 2:271-276, 1999).

In addition, the analysis of another transgenic mouse strain, the PS1(M146L) transgenic mouse, indicates that mutated, but not wild-type, PS1 expression increases brain amounts of β-amyloid 42 and 43 after 5 months (K. Duff et al., Nature, 383:710-713, 1996). Synaptosomes prepared from these transgenic animals exhibited enhanced elevations of cytoplasmic calcium levels following the exposure to the depolarizing agents β-amyloid peptide and a mitochondrial toxin, 3-nitro-propionic acid. (J. G. Begley et al., J. Neurochem., 72:1030-1039, 1999). However, the PS1(M146L) mouse does not present any pathology (K. Duff et. al., Ibid.).

Interestingly, large plaques develop in the cortex and hippocampus of younger transgenic mice with a double mutation, APP(K670N:M671L)/PS1(M146L), at the age of 8-10 weeks. (L. Holcomb et al., Nature Med., 4:97-100, 1998). At the same age, these mice show an increase in the levels of β-amyloid, suggesting that in these mice synaptic modifications occur at earlier stages than in the transgenic models with a single mutation in APP, or PS1. Similarly, the transgenic mice expressing the amyloidogenic carboxy-terminal 104 amino acids of APP showed LTP impairment, in addition to spatial-learning deficit in the Morris water maze, extracellular β-amyloid deposits, gliosis and cell loss in the CA1 regions of the hippocampus at early stages (i.e., 3 months of age) (Nalbantoglu et al., Nature, 387:500-505, 1997).

Several additional models of AD have become available during the last five years. Since even in the fastest model, Alzheimer's Disease pathology does not start before the end of the second month, it has been necessary to wait at least until this age to inject drugs into the animal to assess whether they prevent, reduce or revert synaptic impairment, plaque formation and increase of β-amyloid levels. Such in vivo approaches have high costs in terms of both time and money as a result of housing expenses, premature death, and the large number of mice needed for each study. In vivo models also pose serious hurdles for Alzheimer's Disease or β-amyloid-associated disease drug discovery, as compounds which prevent, reduce, eliminate, or ameliorate Alzheimer's Disease or β-amyloid-associated disease pathology(ies), and which are likely to have treatment value, cannot be rapidly and efficiently screened or tested.

A solution to the above problems is achieved by the present invention which provides a new, fast, efficient and reproducible in vitro method for the screening and testing of compounds for the treatment and therapy of Alzheimer's Disease or β-amyloid-associated diseases.

### SUMMARY OF THE INVENTION

The present invention provides a new method for testing and screening compounds and materials, such as biologicals, drugs, and the like, for efficacy in affecting, treating, or preventing AD or β-amyloid disease (also called amyloid-beta herein)-associated diseases, including, but not limited to, Down's Syndrome. In accordance with this invention, the method involves the use of cultured cells that are established from animal models of Alzheimer's Disease, or β-amyloid-associated diseases. Preferably, the animal models of AD are transgenic mouse models that harbor and express genes whose products are associated with the disease state. The cell cultures are prepared and established from early-stage animals and provide an in vitro cultured cell system comprising neuronal cells to test compounds for their effects on the biochemical and physiological functions of cells whose activity is associated with AD or β-amyloid-associated diseases. The present method provides an economical, reliable and efficient technique for testing and screening that does not require waiting to reach the appropriate stage of development of animals typically used for in vivo or tissue-based systems.

It is an aspect of the present invention to provide a cultured cell system comprising neuronal cells, particularly hippocampal neurons, as well as glial cells, for assaying compounds for their effects on cells and cell processes that are involved during the course of AD or β-amyloid-associated disease. Neurotransmission in the hippocampal cell cultures of this invention exhibits fundamental characteristics of neurotransmission in vivo, or in tissue slices. Thus, biochemical, electrophysiological, optical and immunocytochemical properties of the cultured cells are available as testing parameters to determine a compound's effects. In accordance with particular aspects of the invention, compounds undergoing screening as AD or β-amyloid-related disease effectors, treatments, or preventatives, can be tested using the cell cultures as described to determine if the test compounds re-establish mEPSC frequency and/or re-establish plasticity of the cells to normal values, since mEPSC frequency and plasticity in the cultured cells derived from AD animal models have been found to be unlike those of wild type controls.

Compounds undergoing screening as AD or β-amyloid-related disease effectors, treatments, or preventatives, can also be tested using the cell cultures as described to determine if the test compounds re-establish increase in number of cycling vesicles during plasticity to normal values, since number of cycling vesicles in cultured cells from AD models has been found to be unlike that of wild type controls. Cycling vesicles are vesicles that contain neurotransmitter, release the neurotransmitter during synaptic transmission, and are reabsorbed afterwards.

Compounds undergoing screening as AD or β-amyloid-related disease effectors, treatments, or preventatives, can also be tested using the cell cultures as described to determine if the test compounds re-establish increase in number of clusters for synapsin I and/or other presynaptic proteins during plasticity to normal values, since number of synapsin I and/or other presynaptic proteins in cultured cells from AD models has been found to be unlike that of wild type controls.

In yet another of its aspects, the present invention provides a system for the production and isolation of β-amyloid protein. As β-amyloid has been implicated in AD and β-amyloid related diseases, the culture system of this invention can serve as a source of β-amyloid protein, as well as a screening tool, for example, to screen for β-amyloid toxicity as described herein.

Further objects, features and advantages of the present invention will be better understood upon a reading of the detailed description of the invention when considered in connection with the accompanying figures.

### DESCRIPTION OF THE FIGURES

FIGS. 1A and 1B show murine hippocampal neurons in culture. FIG. 1A: Phase-contrast photomicrograph illustrating a neuron at 10 days after plating (scale bar 25 μm). FIG. 1B: Hippocampal neuron in culture stained with antibodies against the specific neuronal marker, MAP-2. Cell was fixed, stained with antibodies against MAP2, and imaged with a fluorescent microscope (scale bar 15 μm).

FIGS. 2A-2C depict synaptic transmission in cultured murine hippocampal neurons. FIG. 2A shows examples of spontaneously and randomly occurring releases of neurotransmitter from the presynaptic terminal, also known as miniature excitatory postsynaptic currents (mEPSCs), in the cultured hippocampal neurons. FIG. 2B shows superimposed examples of postsynaptic currents, i.e., excitatory postsynaptic currents (EPSCs) (upper panel of FIG. 2B). In contrast to the spontaneous mEPSCs, these currents are evoked by the arrival of an action potential to the presynaptic terminal (middle panel of FIG. 2B). The action potential is due to a 10 msec (ms) step (from −60 mV to −40 mV) depolarization of the presynaptic cell (lower panel of FIG. 2B). The application of the excitatory amino acid receptor antagonist, APV, which blocks N-methyl-D-aspartate (NMDA) receptors, reduces the amplitude of the EPSCs. NMDA receptors are part of receptors for glutamate, the major excitatory amino acid of the central nervous system. CNQX, an antagonist of the remaining glutamate receptors, also known as non-NMDA receptors, blocks the remaining part of the EPSC. FIG. 2C shows the time course of EPSC amplitude before and after potentiation by a high frequency stimulation (100 Hz) (here called tetani) (filled circles). Potentiation was blocked by extracellular application of APV (open circles).

FIGS. 3A and 3B show the results of the genotyping of transgenic animals. DNA was prepared from tail tips after digestion with proteinase K and extraction with phenol/chloroform. PCR was used to identify transgene-positive mice. Electrophoresis through a 2% agarose gel stained with ethidium bromide was used to separate bands which were photographed under UV light. The presence of a band of approximately 190 bp (in lanes 3-6) reveals APP transgenes (FIG. 3A), while the detection of a higher band of approximately 210 bp (in lanes 1 and 3-6) is due to PS1 transgenes (FIG. 3B). Lane designations: L (ladder); 1: DNA from PS1 animal; 2: DNA from wild type (WT) animal; 3; 4; 5; 6: DNA from double transgenic mAPP/mPS1 animals.

FIG. 4 illustrates that basal mEPSC frequency of neurotransmitter is increased in cultures from double transgenic mAPP/mPS1 mice compared with WT mice. Treatment with the E64 inhibitor for 3 to 4 days re-established normal mEPSC frequency. # indicates p<0.05.

FIG. 5 shows that the capability of cells to undergo plastic changes is impaired in hippocampal cultures of double transgenic mAPP/mPS1 mice. Treatment with glutamate no longer produced an enhancement of the basal mEPSC frequency in cultures from double transgenic mAPP/mPS1 mice. The introduction of E64 re-established normal synaptic plasticity in cultured cells.

FIG. 6A-6C shows that the capability of cells to undergo increase in number of cycling vesicles is impaired in hippocampal cultures of double transgenic mAPP/mPS1 mice. FIG. 6A shows a schematic representation of the experimental procedure. FIG. 6B shows examples of cycling vesicles before and after application of glutamate in cultures from wild type (WT) animals and mAPP/mPS1 littermates. FIG. 6C shows cumulative plots derived from all the experiments performed with the application of glutamate in cultures from mAPP/mPS1 mice compared with cultures from WT mice. A brief application of glutamate no longer produced enhancement of number of cycling vesicles in cultures from mAPP/mPS1 mice compared with cultures from WT mice.

FIGS. 7A-D show that the capability to undergo increase in number of immunoreactive clusters for synapsin I is impaired in hippocampal cultures of double transgenic mAPP/mPS1 mice. FIG. 7A shows examples of synapsin 1-immunoreactive clusters (red), in a wild type (WT) control dish (left panel), and in a WT dish fixed 30 minutes after brief application of 200 μM glutamate (right panel). Scale bar=5 μm. FIG. 7B shows average results from experiments as in FIG. 7A-left panel (n=10 dishes per group). After glutamate application, the number of synapsin I-immunoreactive clusters increased significantly compared to control dishes. FIG. 7C shows examples of synapsin I-immunoreactive clusters (red), in a mAPP/mPS1 control dish (left panel), and in a mAPP/mPS1 dish fixed 30 minutes after brief application of 200 μM glutamate (right panel). Scale bar=5 μm. FIG. 7D shows average results from experiments as in FIG. 7C-left panel (n=10 dishes per group). After glutamate application, the number of synapsin I-immunoreactive clusters did not increase compared to control dishes.

FIG. 8 shows Synaptophysin-immunoreactivity is increased in cultured hippocampal neurons from ABAD/hAPP mice. Kjk; k;lj;kl j;klgdfg

FIG. 9 shows GLUR1-immunoreactivity is increased in cultured hippocampal neurons from ABAD/hAPP mice.

FIGS. 10A & 10B show the number of synaptophysin immunoreactive puncta is increased in glutamate treated WT dishes compared to control vehicle treated WT dishes, whereas the number of synaptophysin puncta does not increase in the glutamate treated ABAD/hAPP dishes compared to vehicle treated ABAD/hAPP dishes (FIG. 10A). Similar results were obtained with GLUR1 (FIG. 10B).

FIGS. 11A & 11B show Aβ increases the basal frequency of spontaneous release of neurotransmitter (FIG. 11A) and blocks the glutamate-induced mEPSC frequency increase (FIG. 11B).

FIGS. 12A & 12B show Aβ increases the basal number of active presynaptic boutons (FIG. 12A) and blocks the glutamate-induced increase in the number of active boutons (FIG. 12B).

FIGS. 13A & 13B show Aβ increases the basal number of synapsin I-and synaptophysin-immunoreactive puncta (FIG. 13A) and blocks the glutamate-induced increase in the number of synapsin I-and synaptophysin-immunoreactive puncta (FIG. 13B).

### DESCRIPTION OF THE INVENTION

According to the present invention, cell cultures from an animal model of Alzheimer's Disease (AD), in particular, transgenic mouse models of AD, have been established to serve as screening platforms for testing compounds and materials (e.g., drugs and other biological agents and substances) for their effectiveness in the treatment or prevention of AD or β-amyloiad-associated disease. Beta amyloid forms have been shown to be deposited in the central nervous system (CNS) of patients with AD and Down's Syndrome.

A dissociated cell culture system has been newly developed to investigate the cellular mechanisms of learning in hippocampus, a structure within the brain temporal lobe that is particularly critical for memory storage. The hippocampus is one of the brain areas that is affected by AD. As described herein, the biochemical and physiological properties and the responses of these cell cultures, which comprise hippocampal neurons, and their association with AD and β-amyloid diseases, allow them to serve as testing and screening tools for compounds, e.g., new drugs and biologicals, that can affect and be useful for AD or β-amyloid-associated disease treatment or prevention. The cultures are comprised of neuronal cells (neurons) from brain, preferably, murine neuronal cells, and more preferably, hippocampal neurons as well as glial cells.

Illustrative compounds that can be screened or tested in the method and by utilizing the cell cultures of this invention include, but are not limited to, small molecules, e.g., peptides, proteins (e.g., antibodies); biological agents; chemical compounds, and drugs. Preferably, the compounds are non-toxic and well tolerated for use in the treatment, therapy and prevention of AD or β-amyloid-associated disease. The preferred functional activities or effects of such compounds are to inhibit, block, antagonize, reduce, ameliorate, or eliminate adverse effects or debilitating physiological/biological factors associated with AD or β-amyloid-associated disease. The responses of the cells in the culture system of the present invention can be quantitatively assessed, thereby allowing for a determination of the activities or effects of a compound on biochemical and physiological events occurring in cells that are associated with AD or β-amyloid-associated disease.

The utilization of cell cultures, preferably hippocampal cell cultures, obtained from animal models, particularly, transgenic mouse models, of Alzheimer's Disease is a novel approach to drug identification and discovery related to Alzheimer's Disease treatment and prevention. Cell culture systems have numerous advantages over other preparations, e.g., tissue slices or live adult animals, for evaluating the efficacy and use of compounds in the amelioration, treatment or prevention of AD or β-amyloid associated disease, for ascertaining the benefits of new drugs and compounds that affect AD or β-amyloid associated disease; for discovering new drugs and compounds that can affect, treat, or prevent AD or β-amyloid associated disease; and for assessing the effects of such drugs and compounds. Illustrative examples of these advantages include:
(i) easy access for extracellularly applied drugs and compounds undergoing evaluation or testing, as well as the ability to deliver drugs of biologics intracellularly to either side of the synapse;
(ii) the ability to test drug efficacy after a shorter period of time from birth of an animal that represents an animal model of disease;
(iii) the survival of cells from mutant animals with lethal modifications of DNA;
(iv) the visibility of synapses and cells for electrical and optical measurements;
(v) the opportunity to identify pre- and post-synaptic neurons and to examine the monosynaptic response between them;
(vi) the accessibility to the presynaptic terminal;
(vii) the ability to have and maintain long-term access to cells under a controlled environment for biochemical and genetic manipulation; and
(viii) the simplification of a testing, diagnostic, or evaluation system due to the exclusion of different types of neuronal and non-neuronal cells.

To exploit the above-noted advantages, electrophysiological, optical, immunocytochemical and biochemical methods have been combined with the cell cultures derived and established from transgenic animals according to this invention. In a particular embodiment, cultures from double transgenic mice expressing both the human APP mutation (K670N:M671L) (mAPP), and the human PS1 mutation (M146L) (mPS1) have been employed, i.e., mAPP/mPS1 transgenics. This transgenic mouse strain was obtained by crossing APP mutants as described by K. Hsiao et al. (Science, 274:99-102, 1996) with PS1 mutants as described by K. Duff et al. (Nature, 383:710-713, 1996).

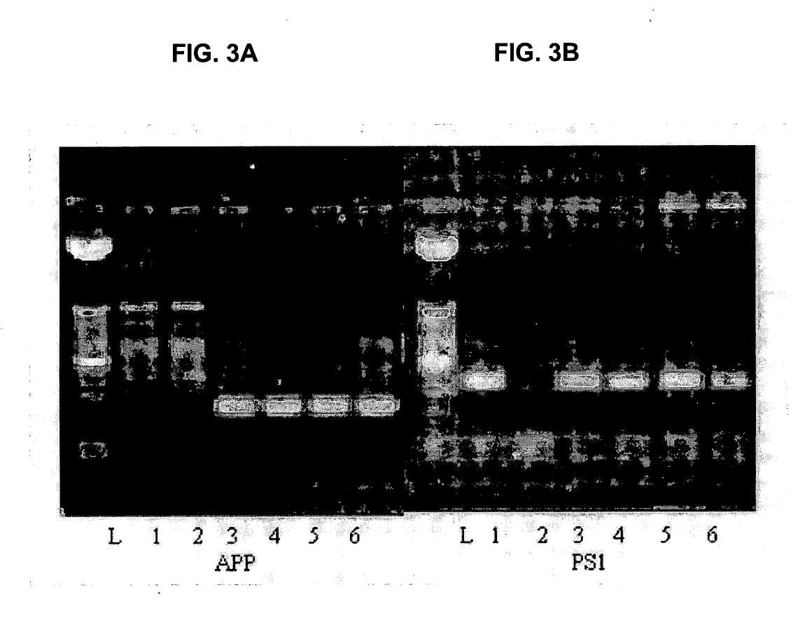

To identify the genotype of the animals, samples of the tails of the mice taken after the dissection of the hippocampus were subjected to polymerase chain reaction (PCR), (FIGS. 3A and 3B). In this embodiment, the cell cultures were prepared from hippocampus, the part of the brain located in the medial surface of cerebral hemispheres. The hippocampi (2 per animal) were obtained from one-day-old double transgenic mAPP/mPS1 mice pups. The significant number of cells obtained from each animal increases the number of drugs and compounds that can be screened using a single mouse. For example, each mouse pup provided enough cells to plate approximately 5 to 6 tissue culture plates containing approximately 1×10⁵ cells per plate. Electrophysiological, optical and immunocytochemical evaluations of the cells were performed and recordings were taken at about 7 to 14 days after plating, instead of at about 8 to 10 weeks that are necessary for live animals.

Thus, the present invention allows the ability to employ cells that provide a faster screening system for drug discovery and efficacy testing than using live animals, in vivo testing, or tissue sections. Even in the fastest animal model of AD or β-amyloid related disease, pathology does not occur before the end of the second month following the birth of the animal. As a result, it has previously been necessary to wait at least this length of time to assess drug efficacy for AD treatment or prevention.

The establishment of a dissociated cell culture system according to the present invention involves the use of plating, culturing, feeding, passaging and maintenance techniques that are known and practiced in the art of cell culture. Briefly, in the present system neuronal cell cultures are established from neonatal animals, preferably from one-day old animals. It was found that older animals, e.g., 2-3 day old mice pups had a generally lower rate of survival of the neurons. Example 1 describes a procedure for obtaining hippocampal cells and establishing neuronal cultures for use according to the present invention.

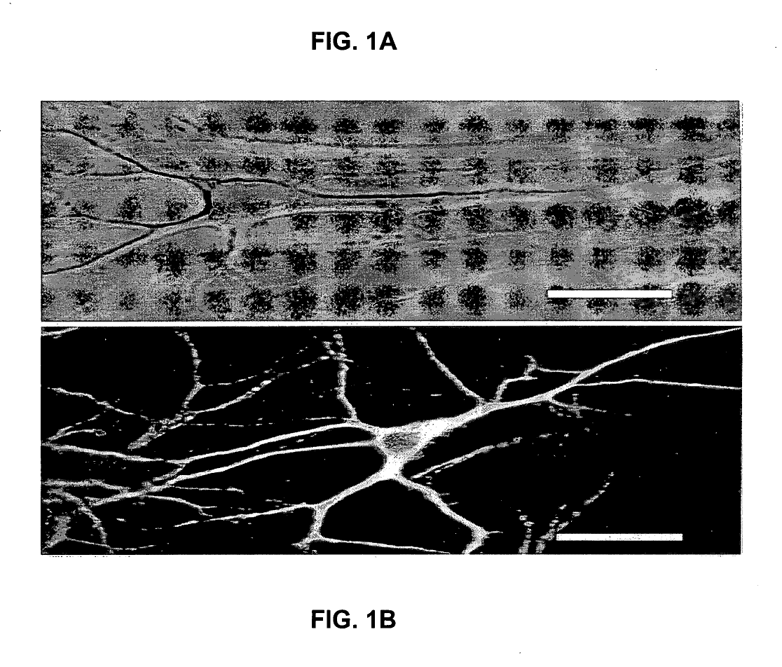

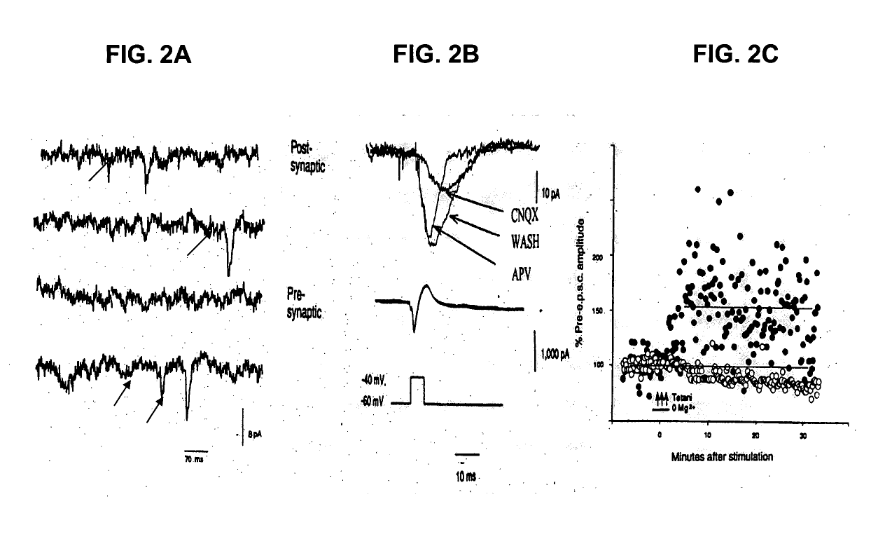

As encompassed by present invention, FIGS. 1A/1B show neurons from a murine dissociated cell culture system developed to investigate and examine the intracellular mechanisms of learning in the hippocampus, which typically shows severe lesions in patients affected by Alzheimer's Disease. Using this in vitro cell culture system, both spontaneous miniature synaptic potentials and evoked postsynaptic potentials have been recorded (FIGS. 2A-2C).

In accordance with the invention, it has been demonstrated that responses in the cultured hippocampal cells (a) are mediated through the activation of NMDA and non-NMDA receptors; (b) show paired pulse facilitation, i.e., a phenomenon involving an increase of the response amplitude when the response is elicited at a short interval after a previous one; and (c) show long-term potentiation (LTP) with similar characteristics to the long-lasting enhancement seen in tissue (e.g., hippocampal) slices or in vivo, thus indicating that Ca²⁺ influx through postsynaptic NMDA receptor channels is required for its induction. Thus, neurotransmission in hippocampal cultures exhibits fundamental characteristics that are highly similar to those in vivo, and/or in tissue slices. As a result, cell cultures can be used in lieu of other preparations to examine such neurotransmission changes.

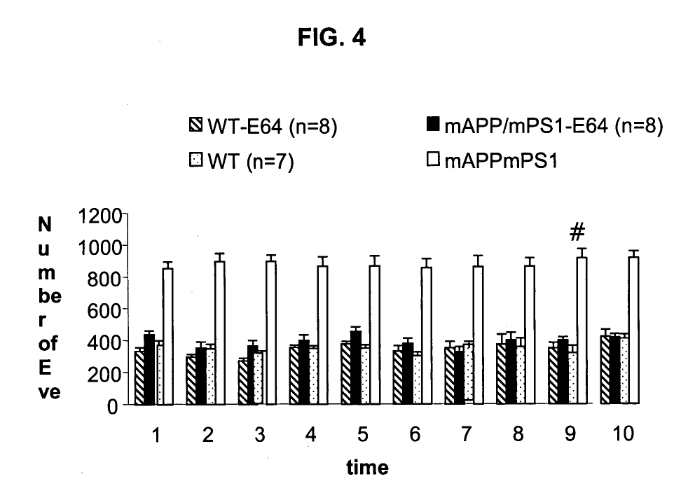

In a particular embodiment in which cultured neurons were obtained from double mAPP/mPS1 transgenic mice, spontaneous neurotransmitter release, also known as mEPSC, was examined to determine whether overexpression of mAPP and mPS1 altered synaptic transmission. Based upon this examination, it was found that basal mEPSC frequency was increased by 157% in cultures from the double transgenic mAPP/mPS1 animals (n=8) compared with cultures from wild type (WT) mice (n=7) (FIG. 4). These novel results indicate that the probability of transmitter release per release site is increased in the double transgenic mice. Interestingly, the mEPSC increase precedes any morphological and behavioral change occurring in live adult animals. Thus, basal mEPSC frequency measured in cell cultures about 7 to 14 days after birth of the animal from which the cells are obtained is one parameter that can be used as a tool, i.e., an in vitro screening or testing system, to assess and evaluate drug efficacy for use in a therapy or treatment for Alzheimer's Disease. In this manner, cultured cells from the double transgenic mAPP/mPS1 animals, or from other transgenic animal models of AD, are useful for testing a compound or substance to determine how the test compound or substance affects basal mEPSC frequency and to specifically ascertain whether the test compound or substance can return the basal frequency of mEPSC in these cells to normal levels, i.e., similar to those of wild type.

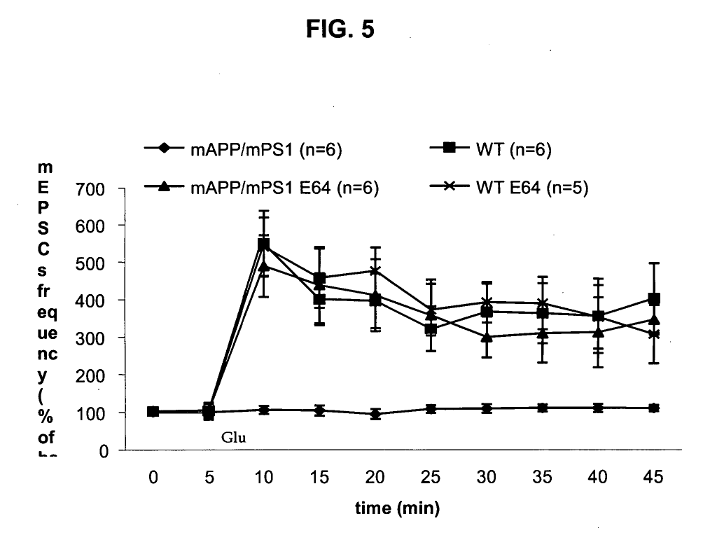

An additional parameter, synaptic plasticity, can also be addressed in the cell cultures according to the present invention. Synaptic plasticity is also altered in the hippocampal neuron cell cultures prepared from double transgenic mice compared to their wild type counterparts. Specifically, it was found that synaptic plasticity was affected following overexpression of the two transgenes, APP and PS1. For example, a brief (approximately 1 minute) application of glutamate (200 μM) did not produce enhancement of mEPSC frequency in cell cultures prepared from the double transgenic mice compared with cell cultures prepared from WT mice (mAPP/mPS1=109.75±5% increase at 45 minutes after glutamate application, n=6; WT=402±95% increase, n=6) (FIG. 5). These data and results indicate that overexpression of the two transgenes blocks the capabilities of cells in the cell cultures from the transgenic animals to undergo plastic changes. In this manner, cultured cells from the double transgenic mAPP/mPS1 animals, or from other transgenic animal models of AD in which synaptic plasticity is adversely affected, are useful for testing a compound or substance to determine whether the test compound or substance can restore the capability of the cultured transgenic cells, which have lost the capability to undergo plastic changes, to regain the ability to undergo plastic changes, akin to wild type.

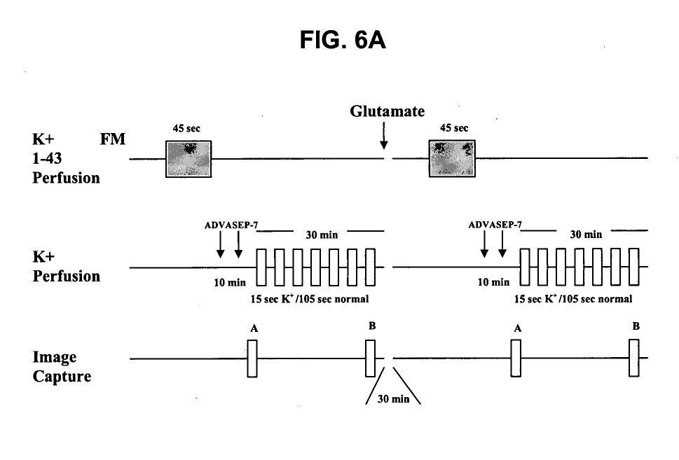

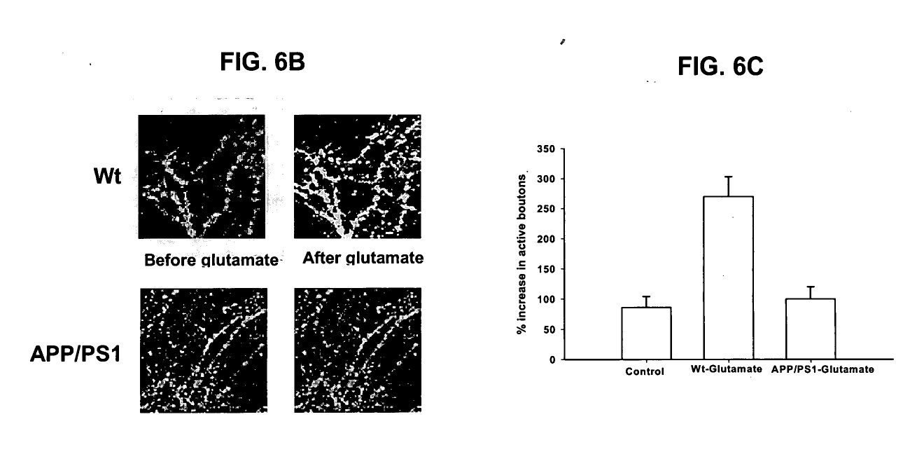

In another particular embodiment in which cultured neurons were obtained from double mAPP/mPS1 mice, the number of cycling vesicles before and after glutamate application were examined to determine whether overexpression of mAPP and mPS1 transgenes altered increase in number of cycling vesicles. Based upon this examination, it was found that brief application (˜30 sec) of 200 μM glutamate in Mg²⁺ free bath solution produced significant increase (268.77±33.12%, n=6) in number of cycling vesicles in cultures from wild-type hippocampal culture (FIG. 6B-C). However, similar application of glutamate failed to increase number of cycling vesicles (100.18±20.2%, n=7) in mAPP/mPS1 culture (FIG. 6B-C). Control experiments revealed that there was no change in number cycling vesicles in both wild-type and mAPP/mPS1 groups without application of glutamate (86±18.6%, n=6) (FIG. 6B-C). These results are entirely consistent with electrophysiological experiments, which demonstrate that synaptic plasticity is impaired in mAPP/mPS1 mice primarily due to presynaptic dysfunction. More importantly, findings with these optical techniques can be adapted in a high throughput type of screening. In this manner, cultured cells from the double transgenic mAPP/mPS1 animals, or from other transgenic animal models of AD in which synaptic plasticity is adversely affected, are useful for testing a compound or substance to determine whether the test compound or substance can restore the capability of the cultured transgenic cells, which have lost the capability to undergo increase in the number of cycling vesicles, to regain the ability to undergo normal changes in cycling vesicles, akin to wild type.

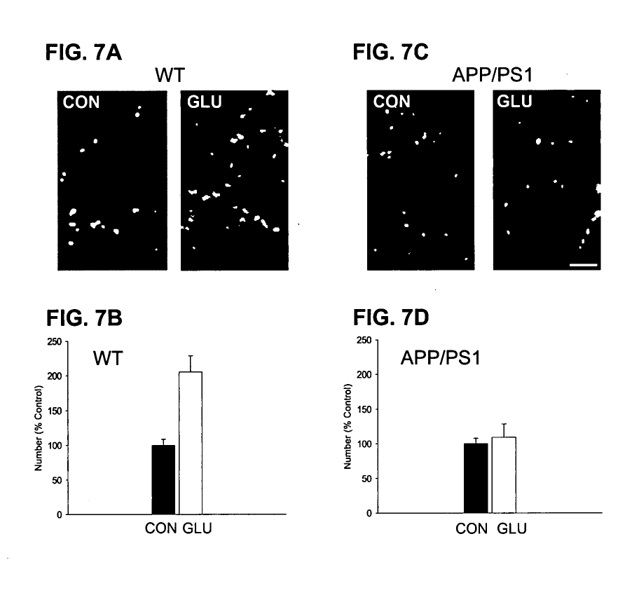

In another particular embodiment in which cultured neurons were obtained from double mAPP/mPS1 mice, the number of immunoreactive clusters for the presynaptic protein, synapsin I, before and after glutamate application were examined to determine whether overexpression of mAPP and mPS1 transgenes altered increase in number of clusters. Based upon this examination, it was found that the number of synapsin I-immunoreactive clusters was increased after glutamate application in wild type cell cultures (average from all such experiments 205.23±23.42% in cultures fixed 5 minutes after the glutamate application), whereas in mAPP/mPS1 cell cultures such increase was not induced (average from all such experiments 109.32±19.11% in cultures fixed 5 minutes after the glutamate application) (FIG. 7). These results provide additional evidence that glutamate-induced plasticity in the presynaptic terminal is impaired following overexpression of APP(K670N:M671L) and PS1(M146L). In this manner, cultured cells from the double transgenic mAPP/mPS1 animals, or from other transgenic animal models of AD in which synaptic plasticity is adversely affected, are useful for testing a compound or substance to determine whether the test compound or substance can restore the capability of the cultured transgenic cells, which have lost the capability to undergo increase in the number of presynaptic protein clusters, to regain the ability to undergo normal changes in presynaptic protein clusters, akin to wild type.

According to the present invention, cell cultures constitute an easy, fast and inexpensive way of screening drugs for use in the treatment or prevention of Alzheimer's Disease. Hippocampal cell cultures as described herein can be established from a variety of different types of relevant animal models of AD, preferably transgenic mice that carry and express genes relating to AD, or β-amyloid-associated disease, and their physiological parameters. Examples of mice suitable for use to obtain the cell cultures according to the present invention include, but are not limited to, the double transgenic mAPP/mPS1 mice, i.e., APP(K670N:M671L)/PS1(M146L) mice (K. Duff et al., Nature, 383: 710-713, 1996), as employed herein.

Further nonlimiting examples of suitable animals to use for establishing the cell cultures as described include APP₇₅₁ mice (Moran et al., Proc. Natl. Acad. Sci. USA, 92:5341-5, 1995); APP(K670N:M671L) mice (K. Hsiao et al., Science, 274:99-102, 1996); transgenic mice expressing the amyloidogenic carboxy-terminal 104 amino acids of APP (Nalbantoglu et al., Nature, 387:500-505, 1997); APP (V717F) mice (Hsia et al., Proc. Natl. Acad. Sci. USA, 96:3228-3233, 1999); mice overexpressing RAGE and a mutant form of APP (V717F, K670N,M671L) (O. Arancio et al., Soc. Neurosci. Abstr., 27:860.18, 2001); and mice overexpressing ABAD and a mutant form of APP (V717F, K670N,M671L) (Yan et al., Soc Neurosci. Abstr., 26:804, 2000). It is to be understood that the present invention also embraces other mouse models of AD and β-amyloid associated diseases, which are also suitable for use as starting material for the cell cultures and screening system of the present invention.

Electrophysiological, optical and immunocytochemical properties of cultures neurons derived from AD transgenic models can be exploited to screen in an easy, fast, and inexpensive way drugs for use in the treatment or prevention of AD. However, these are not the only properties that can be exploited in these culture systems. Another embodiment of our invention provides a system for biochemical detection through techniques such as western blot, microarrays, proteonomics, etc, of genes and proteins that are triggered during the process of learning and other clinical features of AD. Examples of genes and proteins are: cAMP-response element binding protein (CREB), CREB-dependent gene, CCAAT enhancer binding protein β(C/EBP), C/EBP downstream target genes, cAMP-dependent protein kinase, and cGMP-dependent protein kinase. An advantage of these methods is the possibility of adapting them in a high-throughput screening.

Yet another embodiment of the present invention relates to a key pathological feature of Alzheimer's Disease, i.e., elevated concentrations of the β-amyloid peptides. For example, the amounts of the β-amyloid peptides 42 and 43, have been reported to be elevated in AD transgenic mice (P. F. Chapman et al., Nature Neurosci., 2:271-276, 1999; K. Duff et al., Nature, 383:710-713, 1996; L. Holcomb et al., 1998, Nature Med., 4:97-100; K. Hsiao et al., 1996, Science, 274:99-102). Observations of primary cultures have shown that the levels of these peptides increase as the cultures become older. (C. Haass et al., Nature, 359:322-325,1992; P. Seubert et al., Nature, 359:325-327, 1992). The concentrations of β-amyloid peptides 42 and 43 were measured using an enzyme-linked immunosorbent assay (ELISA) (Janus et al., Nature, 408:979-982, 2000) at different stages (i.e., time in culture) of the primary cell cultures.

Interestingly, highly elevated levels of the β-amyloid 40 and β-amyloid 42 peptides were found in hippocampal cell cultures of this invention obtained from double transgenic mice compared with WT (control) animals. More specifically, at 12 days after plating the cells, human β-amyloid 40 and 42 levels were 95±18 fmol/ml and 30.5±8 fmol/ml, respectively, (n=6 dishes). In contrast, in control animals, the levels of these β-amyloid peptides were undetectable (n=5). In a separate series of experiments performed with the same methods as for the hippocampal cultures, the same measurements were repeated on cortical cell cultures derived from double transgenic mice and WT (control) animals. More specifically, at 8 days after plating, human β-amyloid 40 and β-amyloid 42 levels were 249±28 fmol/ml and 79±15 fmol/ml, respectively, (n=3 dishes). In contrast, in WT animals, the levels of these β-amyloid peptides were undetectable (n=2). At 9 days after plating, the β-amyloid peptide levels were 353±10 fmol/ml and 183±7 fmol/ml, respectively, in the mAPP/mPS1 mice (n=5 dishes), while the WT mice showed undetectable values for each of these peptides. On the 10^{th} day, the levels of the two β-amyloid peptides were 313±5 fmol/ml and 160±7 fmol/ml, respectively, in mAPP/mPS1 mice (n=5 dishes), while the levels of the β-amyloid 40 and 42 peptides were undetectable in WT mice. The property of these cells of producing β-amyloid can be exploited in two ways: (a) for screening drugs that interfere with the production of β-amyloid, and (b) as a source for production of β-amyloid that can be isolated and used for behavioral, electrophysiological and toxicological testing.

In one embodiment, if a substance undergoing testing affects the production of β-amyloid by the cells in the culture, or if β-amyloid fails to be produced, degrades following production, or fails to be secreted after the cells are placed in contact with the substance, then the substance can be selected and further tested as an agent that may attenuate β-amyloid production or toxicity, and as a beneficial compound for AD or β-amyloid-associated disease treatment, therapy, or prevention. In addition, toxicity analyses using isolated β-amyloid can be performed to screen substances that can degrade or otherwise disrupt this protein, and thus impair or prevent its ability to cause or be associated with AD or β-amyloid-related disease.

In another embodiment embraced by the present invention, the established cell cultures as newly described herein provide a system for the production and isolation of β-amyloid. During their growth in culture, the cells synthesize and secrete β-amyloid protein into the culture medium, e.g., as described above without limitation for β-amyloid 40 and 42 (see also, Example 1), and thus serve as an in vitro source of naturally occurring β-amyloid protein. Due to its implications in AD and β-amyloid-associated disease, this protein is a valuable resource for investigations, such as diagnosis and testing, related to β-amyloid toxicity, for example.

For guidance without intending to be limiting, the cell cultures typically produce β-amyloid protein in about 8 to 10 days after being placed in culture. About 5.5 to 50,000 fmole/ml of β-amyloid protein can be detected in the cultures, and at least about 5.5 fmole/ml of β-amyloid can be used in an assay (Janus et al., Nature, 408:979-982, 2000; Rozmahel et al., Neurobiology of Aging, 23:187-194, 2002; and P. Mathews et al., J. Biol. Chem., 277: 5299-5307, 2002). Quantification of similar low amounts of the protein can also be achieved using sensitive ELISA immunoassays, for example, using kits commercially available from BioSource International (Camarillo, Calif.). Larger amounts of amyloid-beta protein (e.g., ≧500 fmole/ml) can be isolated by conventional techniques, such as immunoprecipitation and Western Blot, employing specific antibodies, (e.g., 4G8 and 6E10 monoclonal antibodies specific for human β-amyloid peptide commercially available from Signet Laboratories, Dedham Md.), as routinely practiced in the art. Beta amyloid protein is isolated from medium collected from the cell cultures (see, for example, Example 1) to obtain supematant containing the protein.

The toxicity of the isolated and/or purified β-amyloid can be tested electrophysiologically according to this invention by adding the protein to a bath solution containing the cultured cells to observe if there are any electrophysiological changes, such as described herein. In addition, the protein can also be added to the perfusion medium of hippocampal slices (e.g., for about 20 minutes) to test whether the long-term potentiation of the hippocampal cells is impaired (as it should be if the drug is effective). Further, isolated β-amyloid can be injected into live animals to assess if any behavioral or electrophysiological changes occur. To inject the protein, a cannula can be implanted in the lateral cerebral ventricle of the animal (exemplary coordinates: 0.5 mm anterior to bregma and 1.0 mm right of midline). Conditioned medium samples (e.g., approximately 1.5 ml) can be injected (e.g., over about a 2 minute period prior to (e.g., about 10 minutes before) checking the behavior or testing the capability of the animals to show long-term potentiation (D. M. Walsh et al., Nature, 416:535-39, 2002). Any other type of commercially available delivery system can be also used.

Behavioral testing of animals is performed using assessment tests that examine both working and reference memory (D. Diamond, et al., Hippocampus, 9:542-552, 1999—radial arm water maze, working memory; G. W. Arendash, et al., Brain Res., 891:42-53, 2001—Morris maze, reference memory). These tests are generally based on the fact that mice can find the location of a hidden platform in a maze full of water based on spatial cues located in the room where the test is performed. Long-term potentiation (LTP) is tested by implanting electrodes in the skull of live mice (D. M. Walsh et al., Nature, 416:535-39, 2002). These electrodes record the extracellular responses produced in the hippocampus by electrical stimulation. LTP is elicited by a high frequency stimulation (100 Hz), also known as tetanus.

### EXAMPLES

The following examples describe specific aspects of the invention to illustrate the invention and provide a description of the present methods for those of skill in the art. The examples should not be construed as limiting the invention, as the examples merely provide specific methodology useful in the understanding and practice of the invention and its various aspects.

### Example 1

### Cell Cultures

Cell cultures were prepared from one-day-old mouse pups. The hippocampus, located in the medial surface of cerebral hemispheres of the brain, was surgically dissected from the remaining part of the brain under a stereo-microscope. By doing so, other types of cells not belonging to the hippocampus were excluded, while retaining all of the different types of hippocampal cells (both neuronal and glial). Cells were dissociated using enzymatic treatment with 0.25% trypsin (GIBCO BRL: cat #15090-046) in S-MEM (GIBCO BRL: cat #11380-037) for 30 minutes and subsequent trituration. Although trypsin is preferred, other enzymatic treatments (e.g., papain) will not change the outcome of the dissociation.

The cells were plated on glass coverslips (Fisher Scientific, Pittsburgh, Pa.: cat #12-518-105K) previously coated with (10 μg/ml) poly(D-lysine) (Sigma, St. Louis, Mo.: cat #P-7886) for at least 3 hours at 4° C., followed by laminin (1 mg/50 ml) (BD Biosciences, San Diego, Calif.: cat #35-4232) for at least 1 hour in an incubator containing 5% CO₂. At the center of the glass coverslip a ring (Thomas Scientific, Swedesboro, N.J.: cat #6705R12) was placed. Each glass coverslip was contained in a 35×10 cell culture dish (Nalge Nunc, Rochester, N.Y.: No. 153066). Approximately 150 μl of solution containing approximately 100,000 cells were placed inside the ring. Hippocampal cells were grown in medium containing 84% Eagle's minimum essential medium (MEM) (GIBCO BRL, Calif.: cat #12370-037), supplemented to contain 10% heat-inactivated fetal calf serum (HyClone Laboratories Inc., Logan, Utah: cat #SH30070.02), 1.62 mg/l glucose, 1% MEM vitamin solution (GIBCO BRL: cat #11120-052), and 400 μM glutamine (Sigma: cat #G-7513). Cells were kept inside a 5% CO₂ incubator at 37° C. After 24 hours, this medium was replaced by a medium (DMEM) containing 96.5% Neurobasal A (GIBCO BRL: cat #10888-022), 2% B27-nutrient (GIBCO BRL: cat #17504-044), 1% heat-inactivated fetal calf serum (HyClone Laboratories Inc.: cat #SH30070.02), 80 μM glutamine (Sigma: cat #G-7513), 16.67 μl of 6N HCl solution, 125 μM Kynurenic Acid (Sigma: cat #K-3375), 25 μM 5-fluoro-2-deoxyuridine (Sigma: cat #F-0503), 70 μM uridine (Sigma: cat #U-3750) and 5 μM 2-Mercaptoethanol (Sigma: cat #M-6250). This medium was no longer replaced in the following days. Cells plated in this medium in culture developed processes, made synaptic contact and lasted for about 20 to 25 days. For use in experiments for screening and testing compounds and materials as described herein, the cells were typically used on about days 8 through 15 of culture. The cells were used directly from the incubated cultures and were not frozen and thawed. The cell culture media as described above for establishing, growing and maintaining the cells of the present invention can be prepared and stored for about a month at 4° C.

### Electrophysiology

Cultured neurons were voltage clamped with the whole cell ruptured patch technique throughout the experiment. The bath solution contained (in mM): NaCl (119), KCl (5), HEPES (20), CaCl₂ (2), MgCl₂ (2), glucose (30), glycine (0.001), picrotoxin (0.1), at pH 7.3, osmolarity adjusted to 330 mOsm with sucrose. The solution in the whole cell patch electrode contained (in mM): K-gluconate (130), KCl (10), MgCl₂ (5), EGTA (0.6), HEPES (5), CaCl₂ (0.06), Mg-ATP (2), GTP (0.2), leupeptin (0.2), phosphocreatine (20), and creatine-phosphokinase (50 U/ml). Currents were recorded with a Warner amplifier (model PC-501A) (Warner Instrument Inc., CT), and were filtered at 1 kHz. In order to eliminate artifacts due to variation of the seal properties, the access resistance was monitored for constancy throughout all experiments. In order to suppress action potentials, 1 μM tetrodotoxin was added to the bath when recording mEPSCs. They were digitized and analyzed with the mini analysis program (version 4.0) from Synaptosoft, Inc. (GA). A baseline of 10-minutes duration was acquired before inducing LTP through 100 μM glutamate application in Mg²⁺ free solution.

### Vesicle Cycling

Cationic styrylpyridinium dye FM 1-43 has become an established tool for identifying actively firing neurons and for investigating the mechanisms of activity-dependent vesicle cycling in widely different species. These water soluble dyes, which are non-toxic to cells virtually nonfluorescent in aqueous medium, become internalized within recycled synaptic vesicles and the nerve terminals become brightly stained. The amount of FM 1-43 taken up per vesicle by endocytosis equals the amount of dye released upon exocytosis.

One of the methods to induce exocytosis/endocytosis in the neuronal culture is by perfusion of the hyperkalemic solution. As illustrated in FIG. 6A, loading of FM 1-43 is induced by changing the perfusion medium from normal saline bath solution (119 mM NaCl, 2.5 mM KCl, 2 mM CaCl₂, 2 mM MgCl₂, 25 mM HEPES and 30 mM glucose) to hyperkalemic bath solution (31.5 mM NaCl, 90 mM KCl, 2 mM CaCl₂, 2 mM MgCl₂, 25 mM HEPES and 30 mM glucose) with 5 μM FM 1-43 for 45 seconds. The perfusion solution is then changed back to normal bath solution for 10 minutes to wash off the dye from the external medium. ADVASEP-7 (1 mM, CyDex, Inc., Overland Park, Kans.), an anionic cyclodextrin complexing agent is introduced for 60 seconds in the washing bath solution at 1 and 6 minutes of washing for enhanced removal of the dye from the external medium (FIG. 6A). After 10 min wash period, which is sufficient for the complete recycling and repriming of the dye-stained population of synaptic vesicles, an image was then taken to record the loading of FM 1-43 in the synaptic boutons. The culture is then exposed to multiple 15 seconds application of hyperkalemic bath solution (without FM 1-43) to evoke repeated cycles of exocytosis, which facilitate release of the dye from the vesicles. An image is taken after 30 minutes of repeated cycles of exocytosis and washing with normal bath solution. The difference between the images before and after multiple exposures to hyperkalemic solution gives the measure of FM 1-43 stained vesicles.

To study glutamate-induced presynaptic plasticity changes, the culture is exposed to glutamate (200 μM) in Mg²⁺ free bath solution for 30 seconds and then washed out in approximately 1 minute. After 30 minutes of glutamate exposure, the staining and destaining procedure is repeated. NMDA receptor antagonist, d-AP5 (40 μM) and non-NMDA receptor antagonist, CNQX (20 μM) are included in the hyperkalemic solution to block possible recurrent excitation and induction of activity-dependent plasticity. All images were acquired using Nikon D-Eclipse C1 confocal microscope. Cultures were viewed with 60×/1.2 nA water immersion objective. An investigator blinded to experimental conditions obtained quantitative data using NIH Image (v. 1.61). For each image, the intensity of individual puncta and total number of puncta was assessed. FIG. 6B illustrates exemplary cycling vesicles before and after glutamate exposure in cell cultures from wild type (WT) and mAPP/mPS1 littermates. A brief application of glutamate produces enhancement of cycling vesicles in WT but not mAPP/mPS1 cell cultures (FIG. 6C).

### Immunocytochemistry

Glutamate in Mg²⁺-free bath solution, or normal bath solution (control) was added directly to the culture dish and washed out after approximately 1 min by exchanging the solution with phosphate-buffered saline (PBS), (pH 7.4) 2 times. The cultures were fixed starting approximately 30 min after the application of glutamate, or control solution. The cultures were incubated overnight with affinity-purified rabbit anti-synapsin I (Molecular Probes) diluted 1:200. The secondary antibody was goat anti-rabbit labeled with Rhodamin Red diluted 1:500 in 4% goat serum in PBS. Cells were mounted in Vectashield (Vector Labs) and examined by confocal microscope (Nikon D-Eclipse C1). The cells were excited using the 568 nm lines of a krypton-argon laser to image Cy3. Kalman averages of 4 scans were collected for each image. Ten neurons in each culture dish were selected at random and analyzed by an observer who was blind to the experimental treatment. Fields with roughly equal densities of neurites were chosen for analysis. Synapsin I clusters in a representative field (94×142 μm) around the neuron were quantified using a computer program NIH Image (v. 1.61). Individual clusters were identified based on having a fluorescence intensity that exceeded a threshold set above background and a diameter between 0.5 and 5 μm. For all measures, the mean result from the 10 neurons in the dish was normalized to the mean from control dishes in the same culture batch because variability between different culture batches was greater than between dishes in the same batch. FIG. 7 illustrates the effects of glutamate on the number of synapsin I-immunoreactive clusters in WT and mAPP/mPS1 cell cultures. Synapsin I-immunoreactive clusters are more numerous in glutamate-exposed than control cell WT cultures (FIGS. 7A, 7B). By contrast, application of glutamate to mAPP/mPS1 cell cultures did not enhance the number of synapsin I-immunoreactive clusters compared to control (FIGS. 7C, 7D).

### Biochemistry

β-amyloid levels were assayed from supernatant derived from the medium collected from the culture dishes. β-amyloid was produced by the cells in culture after about ten days and was present in the cell culture medium. The supernatant was centrifuged at 5000 rpm for 5 minutes at 4° C. An ELISA method was used, in which β-amyloid was trapped with either monoclonal antibody to β-amyloid 40 (JRF/cAβ40/10) or β-amyloid 42 (JRF/cAβ42/26), and then was detected with horseradish peroxidase-conjugated JRF/Aβtot/17 (Janus et al., Nature, 408:979-982, 2000). The dilution of JRF/Aβtot/17 and samples were optimized to detect β-amyloid in the range of 50 to 800 fmol ml⁻¹. ELISA signals are reported as the mean±s.e.m. of two replicate wells in fmol γ-amyloid per ml of medium (determined with the BioRad DC protein assay), based on standard curves using synthetic β-amyloid(1-40) and β-amyloid(1-42) peptide standards (American Peptide Co., Sunnyvale, Calif.).

### Example 2

### Use of the Cell Cultures of the Present Invention in AD or Beta-Amyloid-Related Disease Drug Screening/Testing

As an example of the utilization of the cell culture system according to the present invention for drug screening or testing, the cysteine protease inhibitor, E64, was tested to determine its capability of re-establishing normal synaptic transmission in cultures from double transgenic animals. E64 (1 μM), (Calbiochem, San Diego, Calif.) was added daily to the culture medium of cultured hippocampal neurons before recording spontaneous release of neurotransmitter using 6 day old cultured neurons.

The basal mEPSC frequency was recorded in cultures from double transgenic mice treated, or not treated, with E64, as well as cultures from WT mice, treated or not treated, with the inhibitor. A decrease of the mEPSC frequency in the treated mAPP/mPS1 mice (435 events/minute) was observed compared with the mEPSC in untreated mAPP/mPSZ1 (851 events/minute), as well as in WT mice (treated WT mice: 332 events/minute; untreated WT mice: 369 events/minute), (FIG. 4). It was also tested whether E-64 was able to rescue synaptic plasticity impairment. The results demonstrated that the mAPP/mPS1 cell cultures treated with E64 had normal synaptic plasticity (346.44±41% at 45 minutes after application of glutamate, n=6), (FIG. 5). These results demonstrate that it is possible to rescue the changes of synaptic transmission due to the overexpression of the mutated genes (APP and PS1) in the culture system by using appropriate drugs.

### Example 3

We have extended the validity of our findings obtained on the mAPP(K670N:M671L)/mPS1(M146L) mouse to another mouse model of Alzheimer's disease, the ABAD/hAPP mouse (the latter a minigene encoding mAPP695, 751 & 770 bearing mutations linked to familiar AD).

We have demonstrated that cultured hippocampal neurons from ABAD/hAPP mice release into the culture medium two major types of Aβ peptides, Aβ40 and Aβ42. Measurements of Aβ levels contained in the medium collected from 10-day-old ABAD/hAPP cultures revealed the presence of both peptides (average values of Aβ40=401.66±81.23 fmol/mg protein, and Aβ42=233.55±29.79 fmol/mg protein, with AP42/40 ratio=0.58±0.02, n=5 dishes). hAPP cultures showed values of Aβ40=260.26±76.98 fmol/mg protein, and Aβ42=137.22±44.57 fmol/mg protein, with Aβ42/40 ratio=0.52±0.02, n=5 dishes). In contrast, cultures from ABAD and WT littermates showed non-detectable levels of human Aβ40 and 42. These results are consistent with previous studies on cultures from mAPP/mPS1 mice, as already described in the prior Examples. To add validity to our findings a recent manuscript has shown that Aβ is increased in primary cortical and hippocampal cultures from Tg2576 mice, which recapitulate the in vivo localization and accumulation of Aβ42 (Takahashi, R. H. et al., J. Neurosci., 24(14): 3592-3599, 2004).

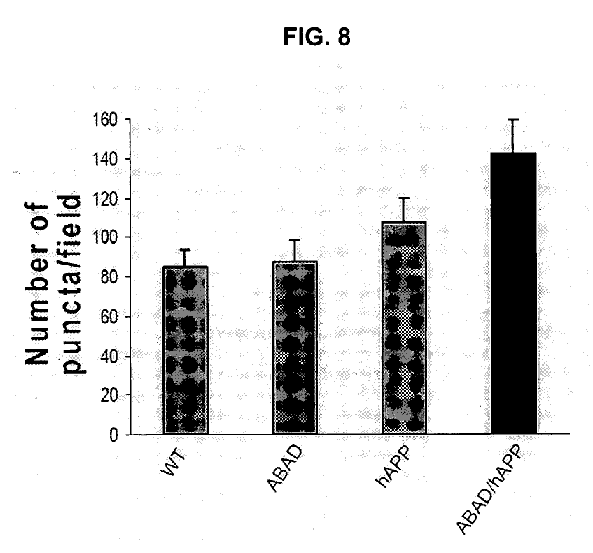

We have also measured immunofluorescence (IF) to presynaptic proteins in cultured hippocampal neurons from Tg hAPP/ABAD, single Tg and nonTg littermates. We have found that basal number of synaptophysin immunoreactive clusters or “puncta” was increased in 10-day old ABAD/hAPP cultures (142.37±16.73 puncta/field, n=8) compared to WT control cultures (64.62±8.94 puncta/field, n=9), ABAD cultures (87.25±11.13 puncta/field, n=8) and hAPP cultures (107.66±12.22 puncta/field, n=9) (FIG. 8). The increase was not associated with change in intensity (98±2.3% of WT controls), or size (102.3±4.0% of WT controls, WT control averages 4.0±0.3 μm²) of synaptophysin puncta, suggesting that the increase was due to gain of new synaptophysin immunoreactive clusters. These results are similar to previous findings in primary hippocampal cultures from the mAPP/mPS1 mouse (see prior Examples) and suggest that Aβ elevation, independently of how it is achieved, produces changes in the distribution and/or expression of presynaptic proteins.

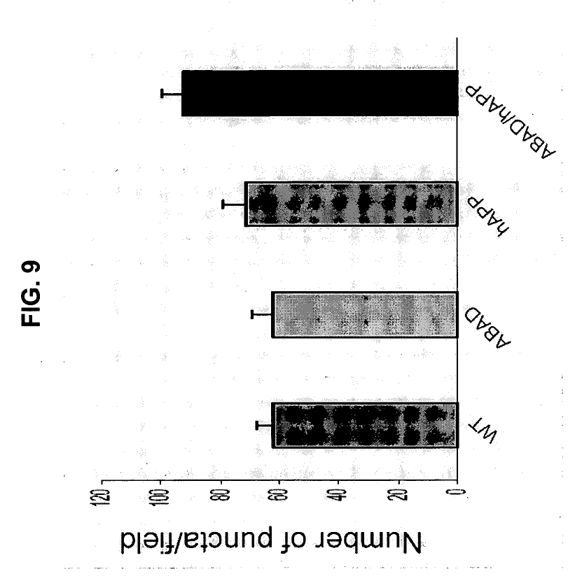

Abnormal expression of AMPA surface membrane receptors has been found in AD patients (Wakabayashi, K. et al. Neurobiol Aging, 20(3): 287-95, 1999). Thus, we have measured immunoreactivity for the postsynaptic protein GLUR1, a subunit of the AMPA receptor. We have found that the basal number of GLUR1 immunoreactive puncta was higher in ABAD/hAPP cultures (92.12±7.48 puncta/field, n=8) compared to WT (61.88±6.04 puncta/field, n=9), ABAD (62.12±7.15 puncta/field, n=8) and hAPP control cultures (71.55±7.44 puncta/field, n=9, FIG. 9), (intensity 96±2.4% controls; size 101.1±2.5% of controls, control size averages 3.9±0.4 μm², data not shown). Thus, ABAD/hAPP overexpression produces also changes in the distribution and/or expression of postsynaptic proteins.

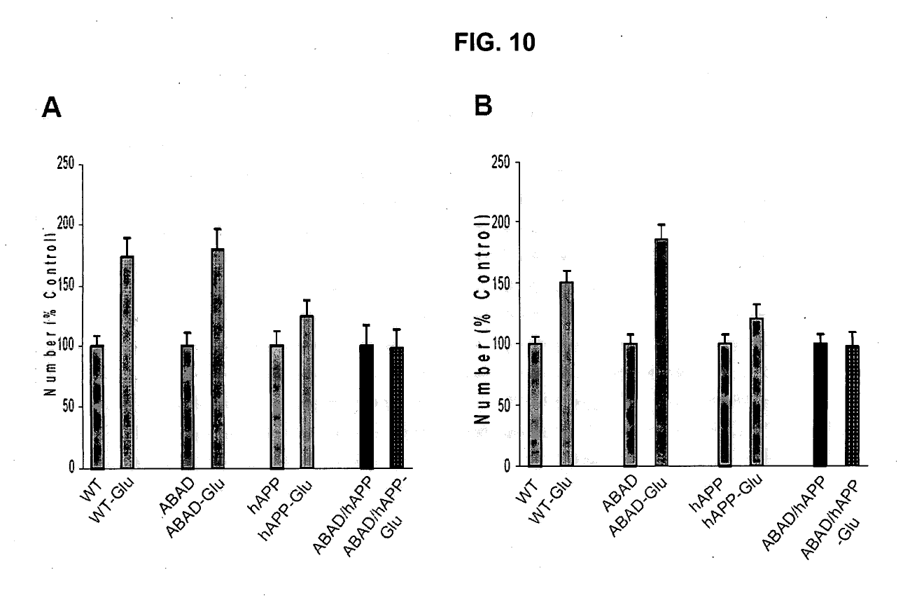

We next tested whether the number of synaptophysin and GLUR1-immunoreactive puncta was increased after glutamate application in ABAD/hAPP cultures. We found that the number of synaptophysin- and GLUR1-immunoreactive puncta was increased following 200 μM glutamate in WT cultures (average from all such experiments 173.26±15.78%/151.07±8.61% synaptophysin/GLUR1 in cultures fixed 30 min after glutamate), whereas in ABAD/hAPP cultures such increase was not induced (98.59±14.61%/97.69±11.98% synaptophysin/GLUR1; FIGS. 10A and 10B). Cultures from hAPP mice showed a very slight increase in synaptophysin/GLUR1 puncta (124.92±13.09%/120.04±11.97% synaptophysin/GLUR1). ABAD cultures showed similar increases in immunoreactivity as WT cultures (179.94±16.36%/185.28±11.91% synaptophysin/GLUR1). These results show that Aβ elevation, independently of how it is achieved, contributes to the block of redistribution of synaptic proteins occurring during synaptic plasticity.

### Example 4

Synaptic changes observed on cultures derived from transgenic mouse models of Alzheimer's disease can be also found in cultures from non-transgenic animals if they are exposed to oligomeric Aβ. Therefore, the use of these changes as a tool to screen drugs that might interfere with the damage of synaptic function by Aβ does not have to be restricted to cultures from transgenic mouse models of Alzheimer's disease. This is demonstrated by the experiments that are described below.

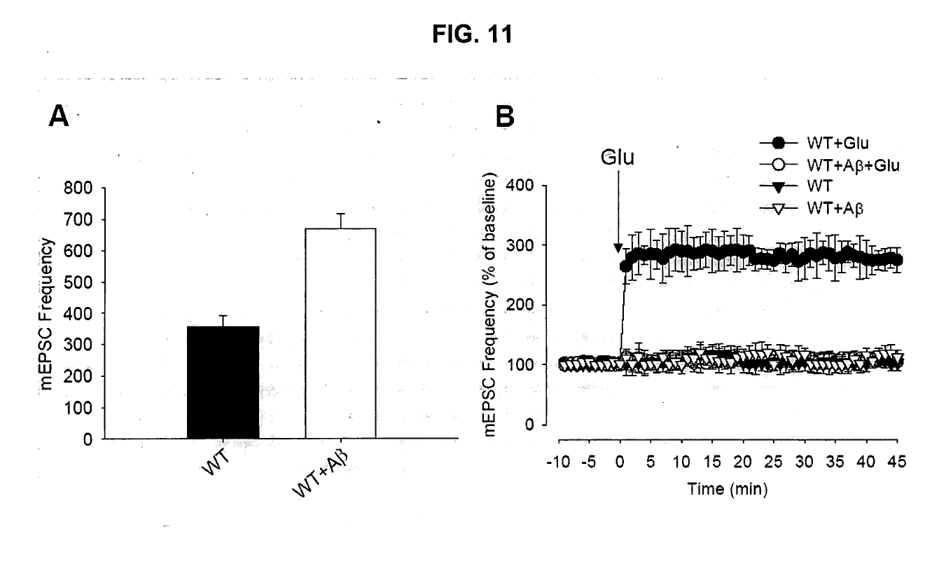

In a series of investigations, we have tested whether application of oligomeric Aβ42 to WT cultures was capable of reproducing the results obtained from hippocampal neurons derived from transgenic animals. We applied 200 pM oligomeric Aβ42 for 24 hours to WT cultures. Then, we tested whether the basal spontaneous release of neurotransmitter was changed in Aβ-treated cultures compared to vehicle-treated cultures. We found that the basal frequency of spontaneous release of neurotransmitter was higher in Aβ-treated WT cultures (671.46±48.28 min-1; n=8), compared to vehicle-treated WT cultures (357.50±34.00 min-1; n=8, p<0.001, t-test, FIG. 11A). We did not find any difference in mEPSC amplitude in Aβ-treated WT cultures compared to vehicle-treated WT cultures (data not shown). These experiments confirm the results obtained with the measurement of basal frequency of spontaneous release of neurotransmitter in transgenic cultures.

To further investigate the effects of Aβ per se on spontaneous release of neurotransmitter, we studied glutamate-induced increase of mEPSC frequency in WT cultures treated with 200 pM Aβ42 for 24 hours. Aβ treatment abolished glutamate-induced increase in mEPSC frequency in WT cultures (104.43±3.05% of pre values at 45 min. after glutamate, n=10, FIG. 11B). In interleaved experiments, 200 μM glutamate was capable of increasing the frequency of spontaneous transmitter release in vehicle treated WT cultures (278.51±20.86% of pre values, n=9, FIG. 11B). Basal mEPSC frequency did not change if vehicle was added to the bath solution, instead of glutamate, to both vehicle-treated (102.00±12.15% of pre values, n=8, FIG. 11B) and Aβ-treated (112.00±11.34% of pre values, n=8, FIG. 1B) WT cultures. There was a significant overall difference between vehicle-treated and Aβ-treated WT cultures following glutamate exposure in a two-way ANOVA with repeated measures (p<0.001). Planned comparisons showed that WT cultures treated with glutamate and either exposed to vehicle or to Aβ for 24 hrs were significantly different at each point (p<0.001). Similar to transgenic cultures, glutamate treatment did not produce any change in mEPSC amplitude in cultures treated with Aβ (data not shown). This result is consistent with the finding that glutamate-induced increase in basal mEPSC frequency is altered in transgenic cultures, and indicates that Aβ per se blocks plastic changes occurring in the spontaneous release of neurotransmitter.

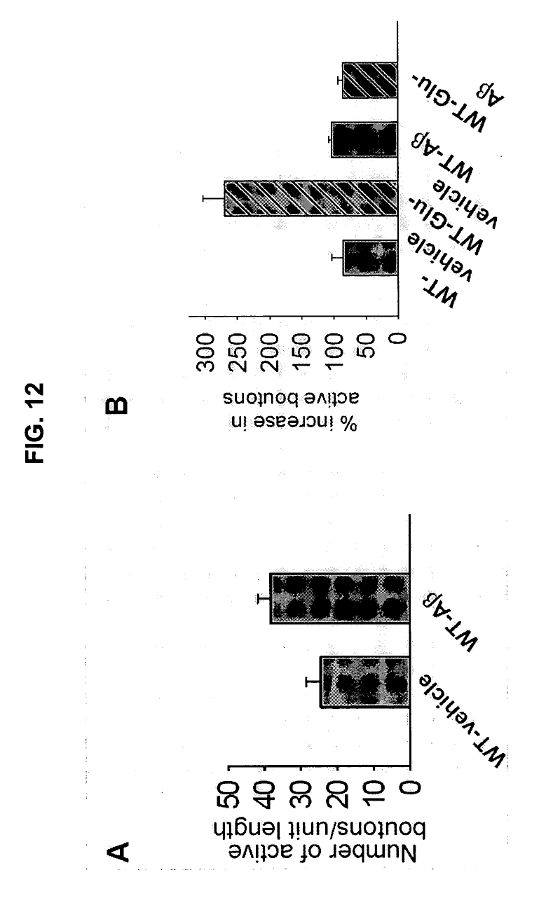

Another finding on cell cultures from transgenic mouse models of Alzheimer's disease is represented by the increase in basal number of functional presynaptic release sites as well as the lack of the glutamate-induced increase in number of functional presynaptic release sites. These tests were performed using FM 1-43. Therefore, we also tested whether these phenomena occurred in non transgenic cultures following exposure to exogenous oligomeric Aβ. We found that the basal number of active boutons was higher in WT cultures treated for 24 hrs with Aβ42 (38.15±3.6 boutons/unit length of neurite; n=15), compared to vehicle-treated WT cultures (24.57±4.0 boutons/unit length of neurite; n=15, p<0.05, t-test, FIG. 12A). In contrast, we did not find any change in the mean fluorescence intensity of the labeled boutons in Aβ-treated cultures. Brief application (˜30 sec) of 200 μM glutamate in Mg²⁺ free medium caused a significant increase in number of active boutons in cultures from vehicle-treated WT animals (269.70±33.12%, n=6, p<0.001, one way ANOVA) ) (FIG. 12B). However, similar application of glutamate failed to increase the number of active boutons in Aβ-treated cultures (86.80±4.6% increase, n=8, p<0.001, one way ANOVA) (FIG. 12B). These results indicate that Aβ alters the basal number of functional release sites as well as their capability to undergo plastic changes.

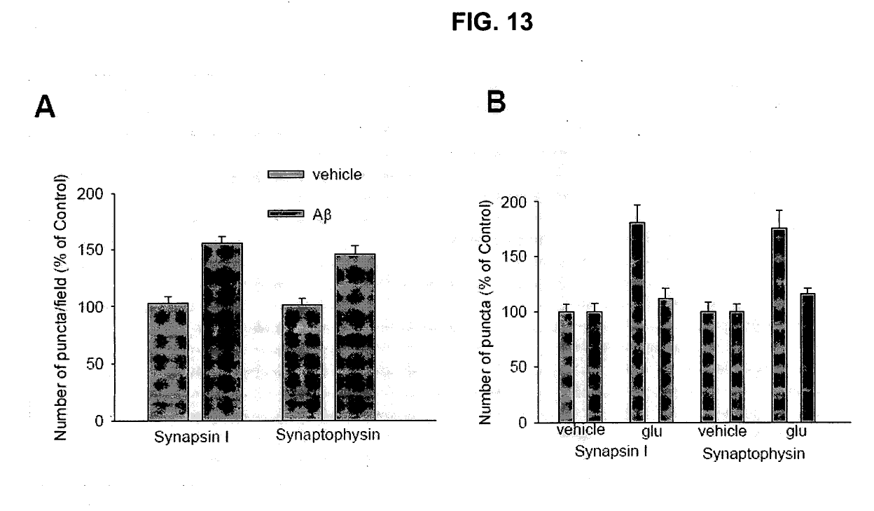

In light of the microstructural changes involving synapsin I and synaptophysin in cultures from transgenic animals, we also tested whether these changes could be induced by the exogenous application of Aβ. We found that the basal number of both synapsin I and synaptophysin immunoreactive clusters or “puncta” was increased when we treated WT cultures with 200 pM oligomeric Aβ42 for 24 hours. The basal number of synapsin I and synaptophysin immunoreactive puncta was higher in WT cultures treated with Aβ than in vehicle-treated WT cultures (average from all such experiments: synapsin 1155.53±6.78% of control, n=11 and 11 dishes; synaptophysin 146.13±6.15%, n=12 and 11; p<0.05 for both synapsin I and synaptophysin, FIG. 13A). We next tested whether the number of synapsin I- and synaptophysin-immunoreactive puncta was increased after glutamate application in Aβ-treated cultures. We found that the number of synapsin I- and synaptophysin-immunoreactive puncta was increased following 200 μM glutamate application in WT cell cultures (average from all such experiments 170.00±16.88%/164.00±16.59% synapsin l/synaptophysin in cultures fixed 30 minutes after the glutamate application, t-test p<0.001 for both presynaptic proteins) (FIG. 13A), whereas in AP42-treated cell cultures such increase was not induced whatsoever (average from all such experiments 111.55±8.97%/115.48±5.76% synapsin l/synaptophysin in cultures fixed 30 minutes after the glutamate application, p<0.05 for both proteins compared to glutamate-treated cultures that were not exposed to Aβ) (FIG. 13B). These findings indicate that Aβ can produce coordinate changes in the distribution and/or expression of presynaptic proteins. These results also indicate that redistribution of presynaptic proteins occurring during synaptic plasticity is blocked by Aβ.

Aβ preparation: Oligomeric Aβ42 was prepared as previously described (Stine et al., 2003). Briefly, the lyophilized peptide (American Peptide) was re-suspended in 100% 1,1,1,3,3,3-hexafluoro-2-propanol (HFIP; Sigma). The solution was aliquoted and the HFIP was allowed to evaporate in the fume hood. The resulting clear peptide films were dried under vacuum in a SpeedVac and stored at −20° C. Twenty-four hours prior to use, the aliquots were added dimethylsulfoxide (DMSO) (Sigma) and sonicated for 10 minutes. Oligomeric Aβ42 was obtained by diluting Aβ42-DMSO into cell culture media, vortexed for 30 seconds and incubated at 4° C. for 24 hours until the use. To induce plasticity 200 μM glutamate in a Mg²⁺-free solution was added for ˜30 sec.

The contents of all patents, patent applications, published PCT applications and articles, books, references, reference and instruction manuals and abstracts cited herein are hereby incorporated by reference in their entirety to more fully describe the state of the art to which the invention pertains.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings.

1. A method of screening or testing a compound for affecting, treating, or preventing Alzheimer's Disease (AD) or β-amyloid-associated, disease, comprising introducing the compound to a cell culture system comprising hippocampal cells from an animal model of AD or β-amyloid-associated disease and determining and/or quantifying, in the cells of the culture system, if the compound affects any one or more of (a) biochemical, (b) electrophysiological, (c) optical, or (d) immunocytochemical parameters associated with AD or β-amyloid-associated disease.

2. A cell culture system comprising hippocampal cells from an animal model of Alzheimer's Disease (AD) or β-amyloid-associated disease, wherein the cells produce β-amyloid protein, wherein said protein can be isolated and used to perform electrophysiological, behavioral, and/or toxicological analyses related to AD or β-amyloid associated disease.

3. A cell culture system comprising hippocampal cells from an animal model of Alzheimer's Disease (AD) or β-amyloid-associated disease, wherein the cell cultures can be used to screen for therapeutic compounds, wherein the therapeutic effect of the compound relates to its ability to interfere with β-amyloid production.

4. A cell culture system comprising hippocampal cells from an animal model of Alzheimer's Disease (AD) or β-amyloid-associated disease, wherein the cell cultures can be used to determine and/or quantify, in the cells of the culture system, if a compound affects any one or more of (a) biochemical, (b) electrophysiological, (c) optical, or (d) immunocytochemical parameters associated with AD or β-amyloid-associated disease.
